(19) United States
(12) Patent Application Publication
(10) Pub. No.: US 2016/0151275 A1
(43) Pub. Date: Jun. 2, 2016

(54) METHOD AND DEVICES FOR MANUFACTURING AND DELIVERING OF AEROSOLIZED FORMULATIONS

(71) Applicants: James Kevin Shurtleff, Provo, UT (US); Jane M. Shurtleff, Provo, UT (US)

(72) Inventors: James Kevin Shurtleff, Provo, UT (US); Jane M. Shurtleff, Provo, UT (US)

(21) Appl. No.: 14/741,372

(22) Filed: Jun. 16, 2015

Related U.S. Application Data
(60) Provisional application No. 62/149,871, filed on Apr. 20, 2015.
(60) Provisional application No. 62/012,486, filed on Jun. 16, 2014.

(51) Int. Cl. A61K 9/00; A61K 45/06; B65B 31/00; A61K 9/12

(52) U.S. Cl. CPC A61K 9/008; A61K 9/124; A61K 45/06; B65B 31/003

(57) ABSTRACT

An aerosolized formulation for use in a metered dose inhaler is disclosed which includes at least one active ingredient, at least one solvent, and at least one propellant. None of the at least one active ingredient, the at least one solvent and the at least one propellant include water.

### CROSS-REFERENCE TO RELATED APPLICATIONS

This application claims the benefit of U.S. Provisional Application No. 62/012,486, filed Jun. 16, 2014, and U.S. Provisional Application No. 62/149,871, filed Apr. 20, 2015, which are hereby incorporated by reference herein in their entireties, including but not limited to those portions that specifically appear hereinafter, the incorporation by reference being made with the following exception: In the event that any portion of the above-referenced provisional applications are inconsistent with this application, this application supercedes said above-referenced provisional applications.

### STATEMENT REGARDING FEDERALLY SPONSORED RESEARCH OR DEVELOPMENT

Not Applicable.

### BACKGROUND

1. The Field of the Present Disclosure.

The present disclosure relates generally to aerosolized formulations, mixtures, products and delivery devices.

2. Description of Related Art

Conventional delivery of pharmaceutically active agents, to the lungs and through the lungs to the body has long existed in the medical community with the use of inhalers. However, inhalers and other similar delivery systems, often deliver inaccurate doses, requiring frequent and/or inconsistent dosing. Frequent inhalation dosing of immediate release formulations can lead to undesired and possibly unsafe or unhealthy levels of the formulation being absorbed by the body of the user.

Effective and efficient formulation delivery, via an inhaler, presents significant challenges. To deliver agents via inhalation, compounds or mixtures must be precisely formulated to ensure that they are deposited to the appropriate part of the lung and to deliver the correct amount of agent over the appropriate period of time. Meeting these objectives require control of key factors such as geometric particle size and density and compatibility with select delivery devices.

Metered dose inhalers have proven to be effective oral and nasal delivery systems which have been used extensively for delivering various medicaments and drugs. Typically, the desired formulation is delivered to the user by a propellant system generally comprising one or more propellants which have the appropriate vapor pressure and which are suitable for oral or nasal administration.

However, an object of the present disclosure is to provide an inhaler system capable of consistent delivery of an aerosolized formulation that enables rapid and consistent absorption into the lungs of a user. Another object of the present disclosure is to identify and provide a formulation of active ingredients, solvents and propellants that maintain solubility and therefore, deliver consistent and accurate dosing of an active ingredient to a user.

The features and advantages of the present disclosure will be set forth in the description which follows, and in part will be apparent from the description, or may be learned by the practice of the present disclosure without undue experimentation. The features and advantages of the present disclosure may be realized and obtained by means of the devices, formulations and combinations particularly pointed out in the appended claims. An understanding of the present disclosure will provide an appreciation of the unique and beneficial combination of the engineering sciences and the medical sciences which result in heretofore unavailable advantages.

### BRIEF DESCRIPTION OF THE DRAWINGS

The features and advantages of the disclosure will become apparent from a consideration of the subsequent detailed description presented in connection with the accompanying drawings in which:

FIG. 1 is a partial cross-sectional view of a metered dose inhaler of the present disclosure;

FIG. 2 is a cross-sectional view of another metered does inhaler of the present disclosure;

FIG. 3 is a picture of a high pressure propellant test apparatus;

FIG. 4 is a close view of the apparatus of FIG. 2;

FIG. 5 is a series of molecular diagrams of active ingredients, solvents and propellants of the present disclosure;

FIG. 6 is a picture of a system for making a metered dose inhaler containing an aerosolized formulation; and,

FIG. 7 is a graph depicting absorption rates and levels of caffeine that is ingested and inhaled.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

It must be noted that, as used in this specification and the appended claims, the singular forms “a,” “an,” and “the” include plural referents unless the context clearly dictates otherwise.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

The present disclosure provides methods and devices for aerosolizing unique formulations in order to deliver various active ingredients directly into alveoli of the lungs of a user, for rapid absorption into the user's blood stream and thereby providing faster benefits of the active ingredients, over conventions liquid or pill consumption by the user.

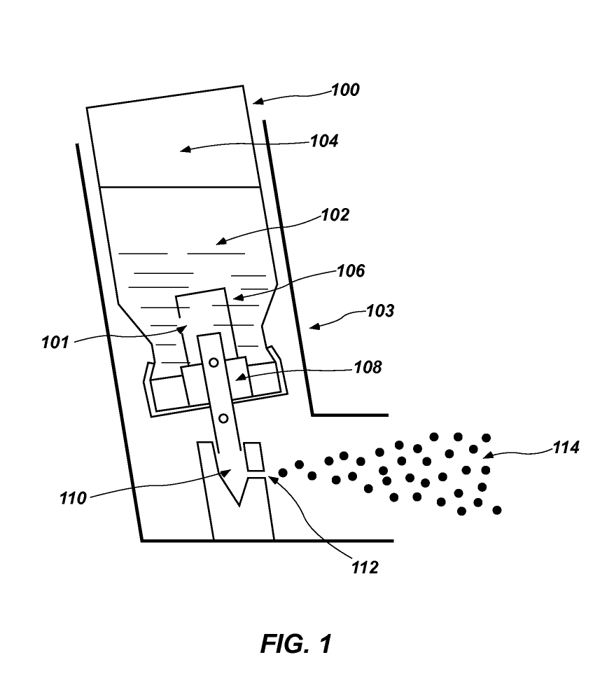

FIG. 1 illustrates a canister 100 used to contain and aerosolize a liquid formulation 102 of the present disclosure. The canister 100 is received within a cavity on an actuator 103, or inhaler. A propellant 104 can be used to facilitate the pressurizing of the liquid formulation 102, by forming a pressurized gas layer above the liquid formulation 102. The liquid formulation 102 fills a retaining cup 106 positioned in the bottom of the canister 100.

When the canister 100 is pushed downward within the actuator 103, a metering chamber 108, which includes a valve, releases a precise, predetermined amount of the liquid formulation 102. The released liquid formulation 102 enters the expansion chamber 110 where the liquid formulation 102 is expanded. The formulation 102 then exits an actuator nozzle 112 forming an aerosolizing formulation 114. The aerosolized formulation 114 is formed of droplets or particles measuring between one (1) and ten (10) micrometers in diameter, for example, 2 micrometers in diameter.

In use, a user can place their mouth over the exit of the actuator 103, press the canister 100 downward against the actuator 103 and inhale deeply to carry the aerosolized formulation 114 into the alveoli of the lungs, where active ingredients in the aerosolized formulation 114 are absorbed rapidly into the blood stream, resulting in a faster perceived benefit of effect of the active ingredient. In another embodiment of the present disclosure,

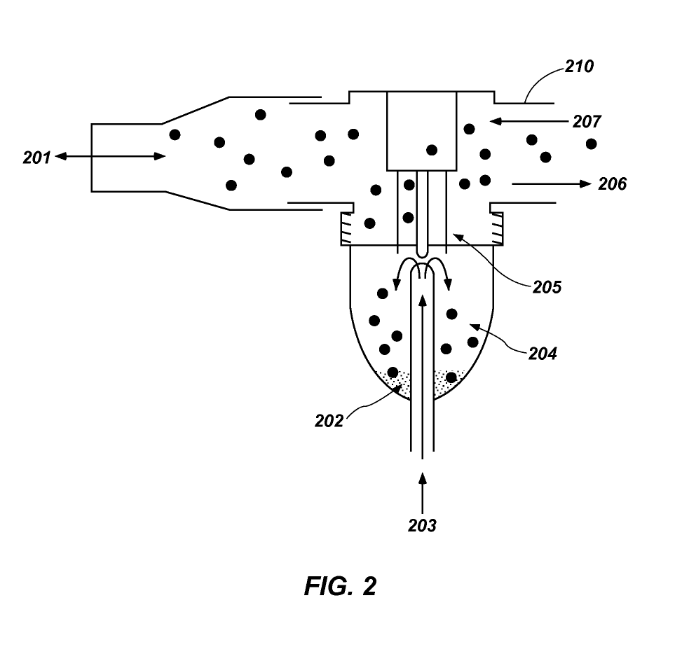

In another embodiment of the present disclosure, as illustrated in FIG. 2, a liquid formulation 202 can be placed inside a nebulizer 204. Compressed air can enter the nebulizer 204 through a tube 203 where the compressed air may be deflected by a baffle or orifice 205, such that the compressed air flows through the formulation 202. As the compressed air flows through the formulation 202 an aerosol of the formulation 202 is formed, with droplets between one (1) and ten (10) micrometers in diameter, for example. Droplets may also be 2 micrometers in diameter.

The user places their mouth over the exit 201 of the nebulizer 204 and inhales deeply. Ambient air enters 207 the nebulizer 204 through an open end 210 of the nebulizer 204. However, one disadvantage of nebulizer 204 is that some of the aerosolized formulation 202 may be lost out 206 the open end 210 of the nebulizer 204. During inhalation, ambient air and the aerosolized formulation 202 are carried into the alveoli of the lungs where the active ingredients in the formulation 202 are absorbed rapidly into the blood stream resulting in the perception of an immediate effect.

In all embodiments of the disclosure, the formulations comprise one or more active ingredients, such as an herbal extract, essential oil, dietary supplement, phytochemical, medicinal compound, or pharmaceutical compound; mixed with inactive ingredients. The inactive ingredients include a co-solvent, such as ethanol, glycerin, or propylene glycol, which dissolve and suspend the active ingredients; a surfactant, such as oleic acid, lecithin, SPAN® 85 (sorbitane trioleate), PVP K25 (polyvinylpyrrolidone), to suspend solid components of the formulation and to assist in the rapid absorption of the active ingredients in the formulation through the mucosal lining of the alveoli, and a hydrofluoroalkane propellant, such as 1,1,1,2-tetrafluoroethane (HFA 134a) or 1,1,1,2,3,3,3-heptafluoropropane (HFA 227), which does not damage the ozone layer, but is necessary to provide the pressurized gas in a metered dose inhaler, as shown in FIG. 1. The propellant is not necessary in formulations used in a nebulizer or other devices used to make an aerosol out of the formulation.

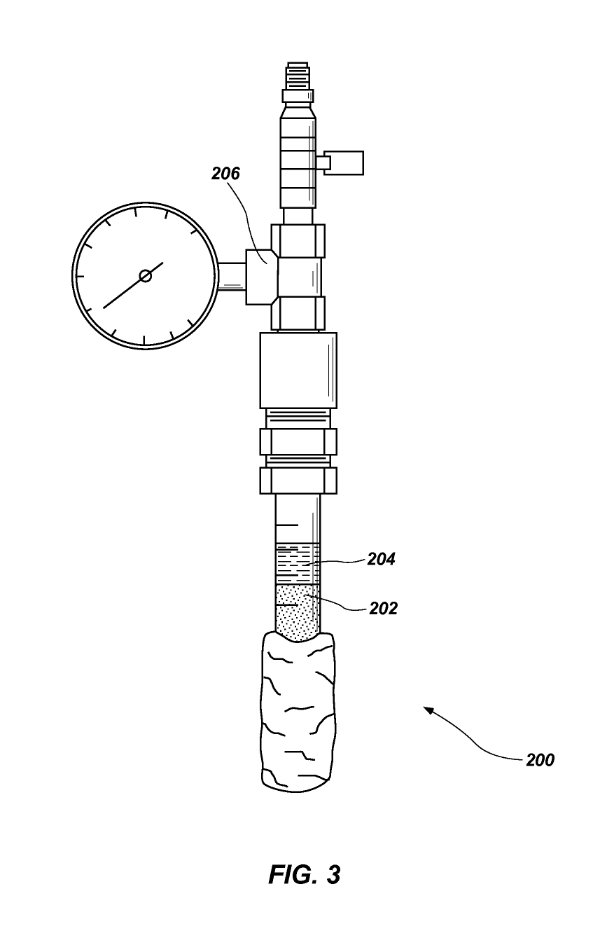

FIG. 3 depicts a transparent, high pressure, test system 200 configured to observe a propellant 202 as a liquid. The propellant 202 can be, for example, HFA 134a (1,1,1,2-tetrafluroethane) or another desired propellant. The propellant 202 can be held within a container 204 with a pressure gauge 206 sealed over the opening of the container 202. As shown in FIG. 3 the pressure gauge 206 indicates that the propellant 102, as shown, is pressurized to slightly more than 70 psig. The propellant 102, such as HFA 134a, must be at substantially 70 psig to remain in liquid form at room temperature.

The propellant 202 is necessary to the process of aerosolizing a liquid. When the propellant 202 and the desired liquid formulation are released from a canister or inhaler, via a metered dose valve, the surrounding ambient air is at a much lower pressure which causes the propellant 202 to immediately vaporize, essentially exploding the less volatile liquid formula into small droplets, which can be less than 5 micron in average diameter. This size of the droplet of the formulation are critical to allow the droplets to be deeply inhaled into the lungs of the user. The ratio of propellant 202 to liquid formulation is also important to produce the desired size of aerosolized droplets. For example, the ratio of propellant 202 to liquid formulation which can achieve the desired droplet size of less than 5 micron in average diameter, can be between 50 volume by percent and 75 volume by percent propellant 202 with the remainder of the volume being the liquid formulation.

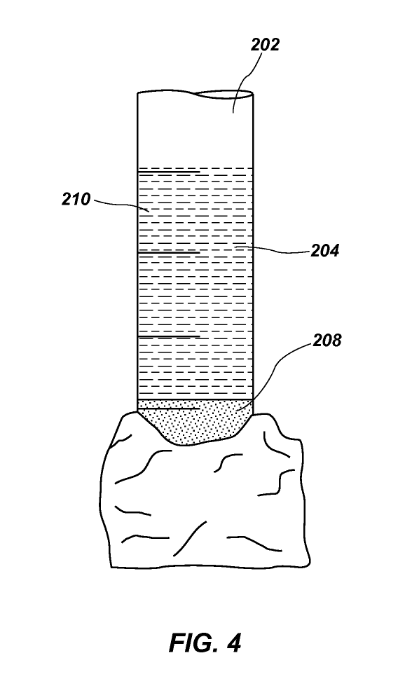

FIG. 4 depicts a 50 volume by percent mixture of propellant 202, HFA134a, with 25 volume by percent water 208 and 25 volume by percent ethanol 210 which separates from the propellant 202. Through careful independent experimentation and analysis, it has been determined that if more than approximately 2 times volume by percent water is present in the liquid formulation, the water causes the propellant 202 to separate from the water present in the liquid formulation.

The mixture of propellant 202 and the liquid formulation must be a uniform and homogeneous to properly form the aerosolized formulation of the desired droplet size as described above. Therefore, to use the desired propellant 202, water cannot be present in the liquid formulation. If water is used in the canister or inhaler, then the more dense propellant, HFA134a, separates and settles on the bottom of the canister and the liquid formulation, rises to the top of the canister. If this separation occurs, then during actuation of the canister or inhaler only propellant 202 will be aerosolized with each actuation with no substantial part of the liquid formulation, particularly the active ingredient, being delivered to the user.

Table 1, below, lists the solubility of an active ingredient, caffeine in this case, in several solvents, including ethanol/water mixtures. It can be noted that caffeine is the most soluble in chloroform. However, for toxicity and environmental reasons, chloroform cannot be reasonably and safely used by a user using an inhaler as disclosed herein.

Caffeine is more than ten (10) times more soluble in a mixture of 60 volume by percent ethanol/40 volume by percent water than it is in ethanol alone. As discussed above, due to the effects of water, the ethanol/water mixture cannot be used, and the solubility of caffeine in ethanol alone is too low to give a desired and appropriate dose of caffeine from an inhaler. Determining that water cannot be used as a component of the liquid formulation was not previously known in the field of inhaler delivery devices and thus the solution to adequately dissolve the liquid formulation with the propellant 202, thereby achieving the desired droplet size, as discussed above, is a key feature of the presently disclosed methods and embodiments for delivering aerosolized formulations.

TABLE 1
(Solubility of Active Ingredients)

| Solvent | Solubility with Caffeine |
|---|---|
| 1 - octanol | Solubility of caffeine in 1-octanol is 0.019M |
| PEG400 | Solubility of caffeine in PEG400 is 0.060M |
| PEG400/water | Solubility of caffeine in PEG400/water (25:75)v |
| (25:75)vol | is 0.078M |
| PEG400/water | Solubility of caffeine in PEG400/water (50:50)vol |
| (50:50)vol | is 0.074M |
| PEG400/water | Solubility of caffeine in PEG400/water (75:25)v |
| (75:25)vol | is 0.060M |
| acetone | Solubility of caffeine in acetone is 0.061M |
| benzene | Solubility of caffeine in benzene is 0.055M |
| carbon | Solubility of caffeine in carbon tetrachloride |
| tetrachloride | is 0.017M |
| chloroform | Solubility of caffeine in chloroform is 0.813M |
| dichloromethane | Solubility of caffeine in dichloromethane |
| | is 0.532M |
| ethanol | Solubility of caffeine in ethanol is 0.029M |
| ethanol/water | Solubility of caffeine in ethanol/water (10:90)vol |
| (10:90)vol | is 0.164M |
| ethanol/water | Solubility of caffeine in ethanol/water (20:80)vol |
| (20:80)vol | is 0.229M |
| ethanol/water | Solubility of caffeine in ethanol/water (30:70)vol |
| (30:70)vol | is 0.293M |
| ethanol/water | Solubility of caffeine in ethanol/water (40:60)vol |
| (40:60)vol | is 0.372M |
| ethanol/water | Solubility of caffeine in ethanol/water (50:50)vol |
| (50:50)vol | is 0.398M |
| ethanol/water | Solubility of caffeine in ethanol/water (60:40)vol |
| (60:40)vol | is 0.470M |
| ethanol/water | Solubility of caffeine in ethanol/water (70:30)vol |
| (70:30)vol | is 0.428M |
| ethanol/water | Solubility of caffeine in ethanol/water (80:20)vol |
| (80:20)vol | is 0.321M |
| ethanol/water | Solubility of caffeine in ethanol/water (90:10)vol |
| (90:10)vol | is 0.158M |
| ethyl acetate | Solubility of caffeine in ethyl acetate is 0.041M |
| methanol | Solubility of caffeine in methanol is 0.048M |
| toluene | Solubility of caffeine in toluene is 0.026M |
| water | Solubility of caffeine in water is 0.105M |

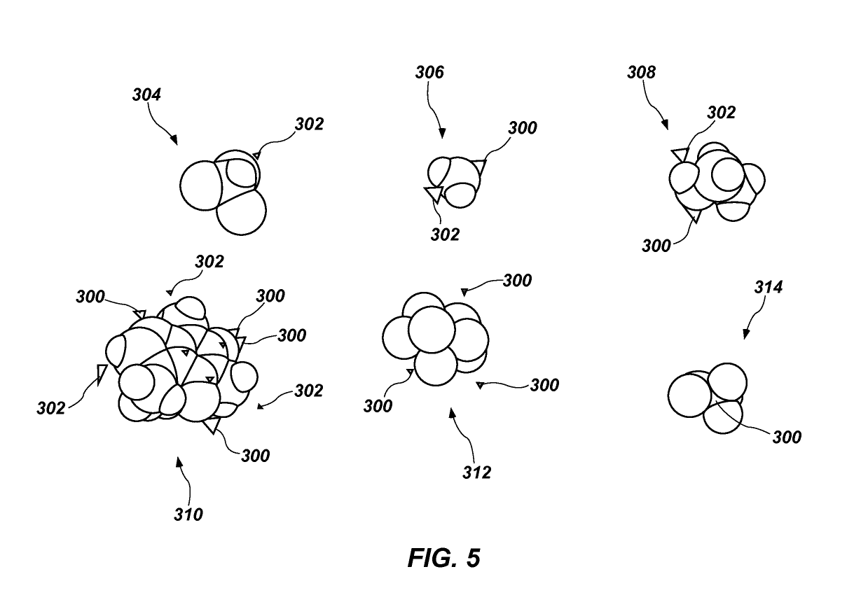

FIG. 5 illustrates a series of three-dimensional diagrams of caffeine molecules and several of the solvents listed in Table 1, above. The diagrams reflect calculations showing the polarity or charge distributions of the molecules. The blue triangles 300 represent a negative potential (−) and the brown triangles 302 represent a positive (+) potential. FIG. 5 illustrates the following molecules with their corresponding polarity: chloroform 304, water 306, ethanol 308, caffeine 310, HFA227 312 and HFA134a 314. In solution chemistry, if solute molecules (e.g., caffeine 310) with a polarity or charge distribution that is similar to the polarity of the solvent molecule, the similarity in polarity will cause solute molecules to dissolve and mix with the solvent molecules, forming a uniform, homogeneous solution.

Separation (insolvency) between a solute molecule and a solvent molecule occurs when the polarity of the solute and solvent molecules are not similar. Based on the diagram of caffeine 310, it can be concluded in accordance with the present disclosure that caffeine 310 has a complex polarity with multiple sites of negative potential. It is not generally known in the field of medical and therapeutic inhaler formula composition that caffeine 310 is substantially soluble in chloroform 304, since chloroform 304 is not significantly polar. In accordance with the present disclosure, caffeine 310 is rendered soluble in chloroform 304, contrary to conventional thinking. As can now be understood from the diagrams if FIG. 5, in accordance with the present disclosure caffeine 310 is soluble in the HFA134a 314. It is noted that HFA134a 314 has a similar polarity and size to chloroform 304. In accordance with the present disclosure, caffeine 310 is more soluble in an ethanol/water mixture than either water 306 or ethanol 308 alone, because the ethanol 308 and water 306 polarities interact to make a quasi-molecule that is much less polar, and thus more soluble with caffeine 310, similar to chloroform 304.

In accordance with the present disclosure, using the high pressure test apparatus 200, as shown in FIG. 3, to determine solubility it is determined that 95% ethanol 308 is completely soluble in HFA134a 314 at concentrations up to 50% volume by percent. Additionally, caffeine 310 is soluble in 50% volume by percent ethanol 308 (95%)/HFA134a 314 solution to a concentration of approximately 20 g/L. However, it is preferred that the ethanol 308 concentration or caffeine 310 concentrations not increase more than these proportions due to the solution breaking down and phase separation occurring again. It has been determined that a small amount of water 306 in the ethanol 308 will cause the solution to separate. Therefore an embodiment of the present disclosure will use 100 volume by percent ethanol 308 to mix with the active ingredient, caffeine 310, so that the liquid formulation will remain solvent and delivering caffeine as an active ingredient.

Other active ingredients can be used in embodiments of the present disclosure. For example, inhaled ibuprofen (HC₁₃H₁₇O₂) can be used as a rapid and effective pain reliever. However, ibuprofen is an organic acid, which can cause a burning sensation on the back of the throat when inhaled. In accordance with the present disclosure, the burning sensation is eliminated by neutralizing with a base. Understanding that the typical acid-base reaction produces water as a product, as shown in Equation 1, below.

H⁺ (acid)+OH⁻ (base)→H₂O Eq. 1

As discussed above, water must be avoided in the liquid formulation to prevent separation of the mixture, a base must be used that will not produce water when mixed with an acid. In accordance with the present disclosure most, if not all, acids can be neutralized with potassium ethoxide (KC₂H₅O). This is the potassium salt of ethanol. In other words, any ethoxide mixed with an acid will produce ethanol, not water, as shown in Equation 2, below.

H+ (acid)+C₂H₅O⁻ (ethoxide)→C₂H₅O (ethanol) Eq. 2

Equation 3 shows the reaction of ibuprofen with potassium ethoxide.

HC₁₃H₁₇O₂ (ibuprofen acid)+KC₂H₅O (ethoxide)→KC₁₃H₁₇O₂ (ibuprofen salt)+C₂H₅O (ethanol) Eq. 3

The need to eliminate water from the acid/base neutralization reaction for the reasons discussed above, was not previously known to those in the field of medical and therapeutic inhaler formulations, and therefore the solution of identifying a mixture that produces ethanol instead of water, is a significant feature of the embodiments of the present disclosure.

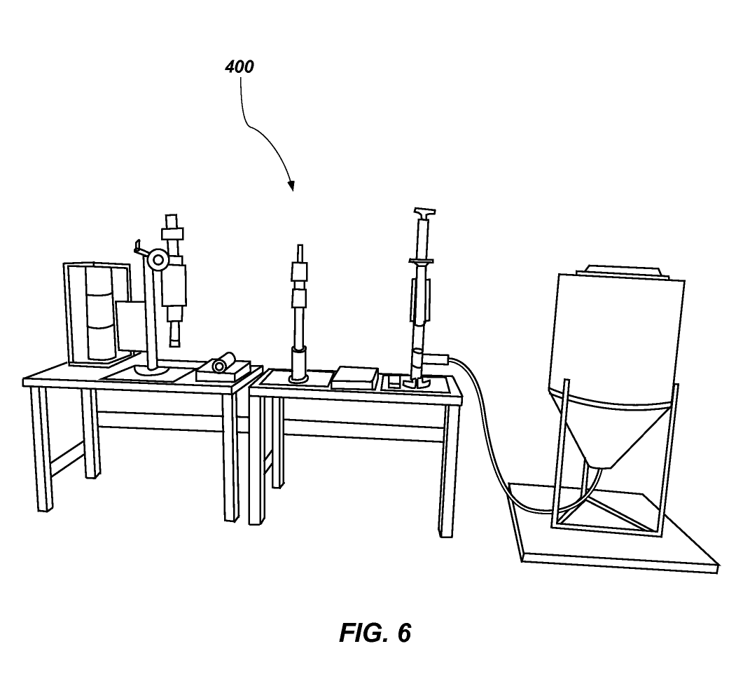

FIG. 6 depicts the system 400 used to make a metered dose inhaler. In Step 1 of making a metered dose inhaler in accordance with the present disclosure, a predetermined amount of formula containing the first solvent, for example ethanol, and the active ingredient, for example caffeine, are added to a canister. In Step 2, a metered dose valve is crimped onto the canister, sealing the canister. In Step 3, a second solvent and propellant, for example HFA 134a, is injected under high pressure into the canister, which completes the desired formulation and inhaler device. Since the HFA134a is not added until Step 3, the active ingredient will not be completely dissolved in the first solvent delivered to the canister in the first step. Therefore, it is necessary that the first solvent and the active ingredient, are continuously mixed to make a uniform suspension (for example, solid caffeine particles in ethanol) that can be carefully metered into the canister, thus insuring the proper and desired concentration of the active ingredient in the final product.

The amount of the active ingredient delivered by each use of the inhaler is critical to making the inhaler effective and safe. If too little active ingredient is delivered than the user will not get the desired results. If too much active ingredient is delivered, the user could receive unsafe and/or unhealthy levels of the active ingredient.

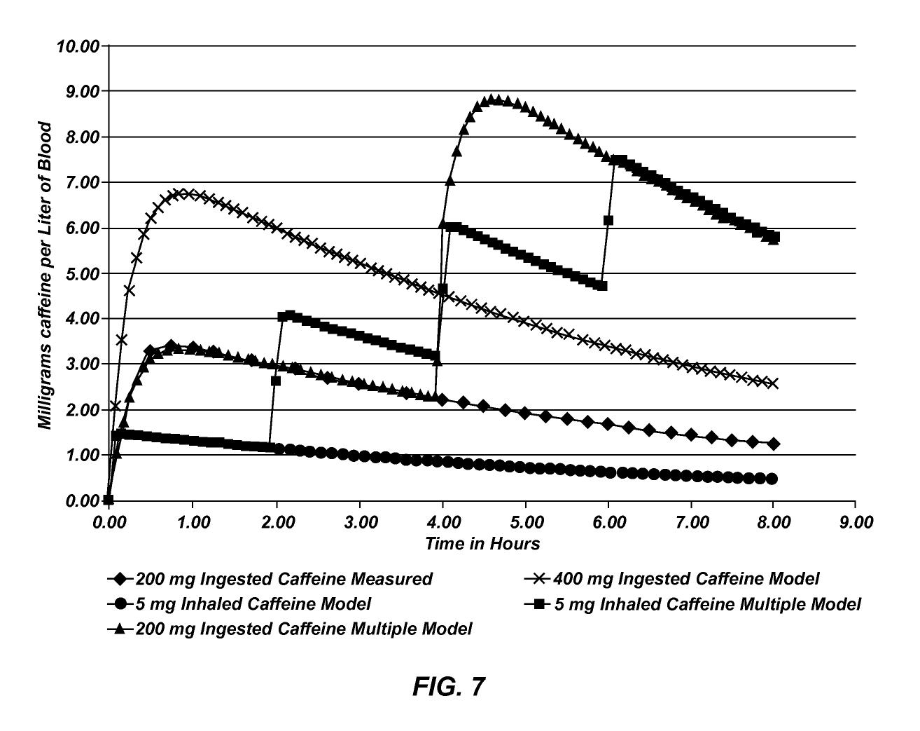

FIG. 7 includes a graph which illustrates the results of an absorption model developed to predict the amount of an active ingredient, in this case caffeine, that would be absorbed into the bloodstream of a user who ingests caffeine or uses the disclosed inhaler to inhale the caffeine. The graph shows the amount of caffeine in the blood (milligrams (mg) of caffeine per liter (L) of blood) upon ingesting (eating or drinking) 200 mg of caffeine. This is an amount of caffeine that is present in a typical energy drink or shot available under the trademarks RED BULL® or MONSTER®. Of particular interest is that when ingested, the amount of caffeine in the blood peaks at approximately 3.5 mg/L after 45 minutes. The line on the graph that indicates “200 mg ingested caffeine multiple model” reflects bioavailability (the amount of caffeine that is absorbed), the absorption rate (the rising part of each curve), and the metabolism rate of caffeine in the liver (the declining part of each curve). It is noted that for a single dose of 200 mg of caffeine, the graph of FIG. 7 closely matches the actual measured data for ingested caffeine.

The “200 mg ingested caffeine multiple model” of FIG. 7 also indicates that the blood caffeine level rises to almost 9 mg/L if a second dose of 200 mg of caffeine is taken four hours after the first dose. This higher level of caffeine in the blood is still safe, although the user likely feels unwanted effects, such as feeling edgy, at this level of caffeine in the blood. The line in FIG. 7 indicating “400 mg ingested caffeine model” shows the predicted caffeine level in the blood would reach 6.75 mg/L in 1 hour after ingesting a single dose of 400 mg of caffeine.

For the disclosed inhaler calculations, it was assumed that 100% bioavailability of the caffeine was absorbed through the lungs. This is likely an overestimate to keep the dosage of the active ingredient, caffeine, low to insure product safety. The actual bioavailability for inhalation is likely 80-90%. For comparison, it has been determined that the bioavailability for ingested caffeine is generally only about 10%. In other words, only 10% by mass of the caffeine actually enters the blood when ingested. The remaining ingested caffeine is excreted. For the inhaled caffeine models in FIG. 7, the absorption rate of caffeine through the lungs was 10× faster than the absorption rate through the stomach and intestines, via ingestion. This means that inhaled caffeine absorption peaks in the blood at about 15-20 seconds.

The “5 mg inhaled caffeine model” of FIG. 7 indicates the results after a user uses the disclosed inhaler once with 5 mg per breath, or use. The inhaled caffeine peaks at approximately 1.5 mg per liter of blood after 15-20 seconds. The “5 mg inhaled caffeine multiple model” indicates the results of taking multiple breaths of caffeine from the disclosed inhaler, every two hours. In the last model, the caffeine concentration in the blood steps up after each dose, but it is still lower than two ingested caffeine doses of 200 mg each. Although the caffeine concentration in the blood is lower when inhaled, than with ingested caffeine, the effects of the caffeine can be felt significantly faster, in about 15-20 seconds. This indicates that inhaled caffeine is much safer than ingested caffeine, because the concentration is lower, while the effects of the caffeine are still felt quickly.

Although the only active ingredient discussed above is caffeine, it has been contemplated that a variety of different active ingredients may be used with the disclosed method and devices, while also using the same disclosed method of providing a solvent, homogenous, aerosolized formulation. For example, the following active ingredients can also be used instead of, or in combination with, caffeine: diphenhdyrameine or other sleep aid, ibuprofen, acetaminophen, or other pain reliever, beta-alanine, B-vitamins, appetite suppressants, calming additives and relaxation aids, caffeine/glucose (dextrose), libido enhancers, cannabidiol, dimenhydrinate or meclizine HCl or other anti-nausea aid, vinpocetine or other study aid, inositol, memory enhancers, oxytocin, dopamine, mucuna pruirns, or other mood enhancers, flavored carbon dioxide, adrenaline, creatin, or ephedrine, nicotine, essential oils, pure oxygen, testosterone or other prescription and non-prescription medications and/or therapeutic ingredients.

In a further embodiment of the present disclosure the aerosolized formulation may include a propellent: HFA134a, 1,1,1,2 tetrafluroethane—50% by volume, 8.6 grams per inhaler; a first solvent: 95 volume % grain neutral spirits, ethyl alcohol, ethanol, 49% by volume, 5.6 grams per inhaler; a second solvent: 100 volume % glycerin, glycerol—1% by volume, 0.18 grams per inhaler; an active ingredient: caffeine, saturated solution or 17.3 grams/liter, 0.25 grams per inhaler, 2.6 milligrams per breath; and a flavoring: cinnamon oil, 1 drop of 0.028 grams per inhaler.

In another embodiment of the present disclosure, the propellant can range from 40 vol %-60 vol % with a corresponding range in the solvent (e.g. alcohol) of 60 vol %-40 vol %. The second solvent (e.g. glycerin) can be 0 vol %-5 vol %, and no more than 5 vol %, for example.

In addition, the propellant and solvent are the key to dissolving the active ingredient to make a single phase, homogenous liquid solution that can be aerosolized. Solubility of the various active ingredients is the key to making the aerosol inhaler function efficiently and effectively, so that a user can inhale small aerosol particles less than 10 micron in diameter, for example, less than 5 micron in diameter.

In another exemplary embodiment of the present disclosure the aerosolized formulation may include, a propellent: HFA134a, 1,1,1,2 tetrafluroethane—50% by volume, 8.6 grams per inhaler; a first solvent: 95 volume % grain neutral spirits, ethyl alcohol, ethanol, 49% by volume, 5.6 grams per inhaler; a second solvent: 100 volume % glycerin, glycerol—1% by volume, 0.18 grams per inhaler; an active ingredient: diphenhydramine HCL—16.7 grams/liter concentration, 0.24 grams per inhaler, 2.5 milligrams per breath; and a flavoring: citrus flavoring, 1 drop of 0.028 grams per inhaler.

In another exemplary embodiment of the present disclosure the aerosolized formulation may include, a propellent: HFA134a, 1,1,1,2 tetrafluroethane—50% by volume, 8.6 grams per inhaler; a first solvent: 95 volume % grain neutral spirits, ethyl alcohol, ethanol, 49% by volume, 5.6 grams per inhaler; a second solvent: 100 volume % glycerin, glycerol—1% by volume, 0.18 grams per inhaler; an active ingredient: nicotine—2.2 grams/liter concentration, 0.32 grams per inhaler, 0.33 milligrams per breath; and a flavoring: cinnamon oil or menthol flavoring, 1 drop of 0.028 grams per inhaler.

In one embodiment of the disclosure, the formulation contains active ingredients including caffeine, guarna, green tea extract, and/or taurine; dextrose; a flavoring; and other active ingredients in order to give the user a perceived energy boost.

In another embodiment of the disclosure, the formulation contains active ingredients including Hoodia Gordinii, omega 3, 6, 9 fatty acids, caffeine, guarna, and/or green tea extract; dextrose; a flavoring; and other active ingredients in order to give the user the perception of appetite suppression.

In another embodiment of the disclosure, the formulation contains active ingredients including yohimbe extract or L-arginine, glutamate, and yohimbine, caffeine, guarna, and/or green tea extract; dextrose; a flavoring; and other active ingredients in order to give the user a perception of increased sex drive.

In another embodiment of the disclosure, the formulation contains active ingredients including hemp oil extract (primarily CBD and other cannabinoids, with no THC); ashwaganda extract; schisandra extract; L-theanine; a flavoring; and other active ingredients in order to give the user a perception of peace, calm, and serenity, but not sleepiness

In another embodiment of the disclosure, the formulation contains active ingredients including green tea extract, L-theanine; a flavoring; and other active ingredients in order to give the user perceived mental clarity without a perception of excess energy.

In another embodiment of the disclosure, the formulation contains active ingredients include valerian, chamomile, L-tryptophan, L-theanine, melatonin, St. John's wort extract, hops; a flavoring; and other active ingredients in order to give the user the perception of sleepiness.

In another embodiment of the disclosure, the formulation contains active ingredients including hemp oil extract (primarily CBD and other cannabinoids with no THC); chamomile, L-theanine; a flavoring; and other active ingredients in order to give the user the perception of reduced nausea.

It is to be understood that the above-described arrangements are only illustrative of the application of the principles of the present disclosure. Numerous modifications and alternative arrangements may be devised by those skilled in the art without departing from the spirit and scope of the present disclosure are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An aerosolize formulation for use in a metered dose inhaler, comprising:
at least one active ingredient;
at least one solvent; and
at least one propellant, wherein none of the at least one active ingredient, the at least one solvent and the at least one propellant includes water.

2. The formulation of claim 1, wherein the solvent is ethanol.

3. The formulation of claim 2, wherein the active ingredient is caffeine.

4. The formulation of claim 3, wherein the propellant is HFA 134a.

5. The formulation of claim 3, wherein the solvent is 100% ethanol.

6. The formulation of claim 1, wherein the amount of solvent can range from about 40 vol % to about 60 vol %.

7. The formulation of claim 6, wherein the amount of propellant can range from about 40 vol % to about 6 vol %.

8. The formulation of claim 1, wherein the solvent can include a first solvent and a second solvent, wherein the first solvent can range from about 40 vol % to about 60 vol % and the second solvent can range from can range from about 0 vol % to about 5 vol %.

9. The formulation of claim 2, wherein the active ingredient is diphenhydramine HCL.

10. The formulation of claim 2, wherein the active ingredient is nicotine.

11. The formulation of claim 1, wherein the active ingredient is selected from the group consisting of diphenhdyrameine or other sleep aid.

12. The formulation of claim 1, wherein the active ingredient is selected from the group consisting of ibuprofen, acetaminophen, or other pain reliever.

13. The formulation of claim 1, wherein the active ingredient is beta-alanine.

14. The formulation of claim 1, wherein the active ingredient is B-vitamins.

15. The formulation of claim 1, wherein the active ingredient is appetite suppressants.

16. The formulation of claim 1, wherein the active ingredient is libido enhancers.

17. The formulation of claim 1, wherein the active ingredient is cannabidiol.

18. The formulation of claim 1, wherein the active ingredient is selected from the group consisting of dimenhydrinate or meclizine HCl or other anti-nausea aid.

19. The formulation of claim 1, wherein the active ingredient is vinpocetine.

20. The formulation of claim 1, wherein the active ingredient is memory enhancers.

21. The formulation of claim 1, wherein the active ingredient is oxytocin.

22. The formulation of claim 1, wherein the active ingredient is dopamine.

23. The formulation of claim 1, wherein the active ingredient is mucuna pruirns.

24. The formulation of claim 1, wherein the active ingredient is adrenaline.

25. The formulation of claim 1, wherein the active ingredient is ephedrine.

26. The formulation of claim 1, wherein the active ingredient is testosterone.

27. A method of making an aerosolized formulation in a metered dose inhaler, comprising:
providing an inhaler device having a canister with an opening;
measuring a predetermined amount of an active ingredient and a solvent;
adding the desired amount of the active ingredient and the solvent inside the canister;
sealing a metered dose valve onto the opening of the canister; and,
adding propellant inside the canister wherein none of the active ingredient, the solvent and the propellant include water.

28. The method of claim 27, further comprising:
continuously mixing the active ingredient and the solvent immediately prior to measuring the desired amount of the active ingredient and the solvent.

29. The method of claim 27, wherein the solvent is ethanol.

30. The method of claim 29, wherein the active ingredient is caffeine.

31. The method of claim 30, wherein the propellant is HFA 134a.

32. The method of claim 29, wherein the solvent is 100% ethanol.

33. The method of claim 28, wherein the amount of solvent can range from about 40 vol % to about 60 vol %.

34. The method of claim 28, wherein the solvent can include a first solvent and a second solvent, wherein the first solvent can range from about 40 vol % to about 60 vol % and the second solvent can range from can range from about 0 vol % to about 5 vol %.

35. The method of claim 29, wherein the active ingredient is diphenhydramine HCL.

36. The method of claim 29, wherein the active ingredient is nicotine.
